(19) United States
(12) Patent Application Publication
(10) Pub. No.: US 2005/0124980 A1
(43) Pub. Date: Jun. 9, 2005

(54) Port stem marking for catheter placement

(76) Inventor: Scott W. Sanders, Lehi, UT (US)

Correspondence Address: MORRISON & FOERSTER, LLP, 555 WEST FIFTH STREET, SUITE 3500, LOS ANGELES, CA, 90013-1024, US

(21) Appl. No.: 10/727,287

(22) Filed: Dec. 3, 2003

(51) Int. Cl. A61K 9/22

(52) U.S. Cl. 604/891.1

(57) ABSTRACT

An access port having a marking located on the port stem to aid the physician in placing the catheter correctly onto the port stem, wherein a fluid channel is provided to allow liquid from the inner lumen of the catheter to flow into the chamber located inside the access port. In one variation, the marking indicates to the physician the proper distance to advance the port stem into the catheter for optimal connection. A catheter lock or other locking sleeves may be placed over the catheter and port stem connection to secure the catheter on the port stem. Since the preferred location for the placement of catheter may differ depending on the design of the port stem, such a marker may facilitate the placement of the catheter and avoid problems associated with physicians placing the catheter incorrectly, which could potentially result in in vivo leakage from the catheter.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A COMPACT DISK APPENDIX

Not applicable.

### BACKGROUND OF THE INVENTION

The present invention generally relates to a subcutaneously implantable access port. More specifically, the present invention relates to the use of markers or indicia on an outlet stem of the access port (i.e., port stem) to facilitate proper placement of a catheter thereon.

A variety subcutaneously implantable access ports haven been utilized by physicians to deliver fluids to, or to withdraw fluids from the blood stream or other subcutaneous cavities inside a patient. One example of such an access port includes a needle-impenetrable housing, which encloses one or more fluid cavities and defines for each of such fluid cavity an access aperture communicating through the housing on the side thereof, which is adjacent to the skin of the patient when the access port is implanted in the body of a patient. A needle-penetrable septum is received in and seals the access aperture. An exit passageways located in a port stem communicates, with the fluid cavities for dispensing medication there from to a predetermined location in the body of the patient through an implanted catheter attached to the access port. Typically, the catheter is connected to the access port by placement of the proximal end of the catheter over the port stem. A locking sleeve or ring may be placed over the catheter at the proximal region of the catheter to secure the catheter on the port stem.

Examples of various access ports and catheter locking mechanisms are disclosed in U.S. Pat. No. 4,772,270, titled “INSEPARABLE PORT/CATHETER TUBE ASSEMBLY AND METHODS” issued to Wiita et al., dated Sep. 20, 1988; U.S. Pat. No. 5,632,729, titled “CATHETER CONNECTOR” issued to Cai et al., dated May 27, 1997; U.S. Pat. No. 4,929,236, titled “SNAP-LOCK FITTING CATHETER FOR AN IMPLANTABLE DEVICE” issued to Sampson, dated May, 29, 1990; U.S. Pat. No. 4,963,133, titled “CATHETER ATTACHMENT SYSTEM” issued to Whipple, dated Oct. 16, 1990; U.S. Pat. No. 5,045,060, titled “IMPLANTABLE INFUSION DEVICE” issued to Melsky et al., dated Sep. 3, 1991; U.S. Pat. No. 5,129,891, titled “CATHETER ATTACHMENT DEVICE” issued to Young, dated Jul. 14, 1992; U.S. Pat. No. 5,137,529, titled “INJECTION PORT” issued to Watson et al., dated Aug. 11, 1992; U.S. Pat. No. 5,312,337, titled “CATHETER ATTACHMENT DEVICE” issued to Flaherty et al., dated May, 17, 1994; U.S. Pat. No. 5,360,407, titled “IMPLANTABLE DUAL ACCESS PORT WITH TACTILE RIDGE FOR POSITION SENSING” issued to Leonard, dated Nov. 1, 1994; U.S. Pat. No. 5,399,168, titled “IMPLANTABLE PLURAL FLUID CAVITY PORT” issued to Wadsworth, Jr. et al., dated Mar. 21, 1995; U.S. Pat. No. 5,833,654, titled “LONGITUDINALLY ALIGNED DUAL RESERVOIR ACCESS PORT” issued to Powers et al., dated Nov. 10, 1998; U.S. Pat. No. 6,113,572, titled “MULTIPLE-TYPE CATHETER CONNECTION SYSTEMS” issued to Gailey et al., dated Sep. 5, 2000; each of which is incorporated herein by reference in its entirety.

Once the access port and the catheter have been implanted beneath the skin of a patient, quantities of medication or blood may be dispensed from the fluid cavity by means of a non-coring needle passed through the skin of the patient and penetrating the septum into the fluid cavity. This medication may be directed to the distal end of the catheter to an entry point into the venous system of the body of the patient. Blood may also be withdrawn for sampling from the body of the patient through such an access port by piercing the skin of the patient and penetrating the septum with a non-coring needle and applying negative pressure thereto, which causes blood to be drawn through the catheter into the fluid cavity covered by the pierced septum and then out of the body of the patient through the needle. To prevent clotting thereafter, the withdrawal route may be flushed with a saline solution or heparin using again a non-coring needle piercing the skin of the patient and the septum in the same manner as if a medication were being infused.

Both intermittent and continual injections of medication may be dispensed by the access port. Continual access may involve the use of a non-coring needle attached to an ambulatory-type pump or gravity feed bag suspended above the patient. The ambulatory-type pump or the gravity feed bag continually delivers the medication or fluid through the needle to the fluid cavity in the access port and from there through the catheter to the entry point into the venous system.

One common problem encountered in the use of access ports relates to the process of connecting the catheter to the access port during the implantation of the access port. The connection is most commonly accomplished by placement of the proximal portion of the catheter over a port stem protruding from the housing of the access port. However, it is generally difficult to determine the amount of engagement of the catheter onto the port stem. For example, some catheter connection systems do not allow visual verification of attachment. In other designs where the physician can visualize the catheter connection, it is generally up the physician to independently determent the proper placement of the catheter over the port stem. As the result, either due to over-insertion or under-insertion of the port stem into the catheter, leakage and failure can occur.

The optimal location for the catheter to be placed over the port stem is determined by the design of the port stem (e.g., location of the barbs on the port stem), the design of cathlock (i.e., catheter lock), ring or sleeve which may be placed over the catheter to secure the catheter over the port stem, and the catheter tubing design and material. In addition, the optimal location for placement of the catheter may vary depending on the access port design. Because access ports with varying designs are used in today's medical practice, it is not unusual for implanted access ports to fail due to improper connection between the port stem and the catheter. In some cases, this is due to doctors who advanced the catheter too far on the stem, and in some cases this is due to doctors who do not advance the catheter far enough on the stem. When the catheter is advanced too far on the stem, there is potential for breakage of the catheter due to pinching and other forces caused by uneven distribution of compression force. The catheter might also bunch up under the cathlock or locking sleeve, thus preventing the cathlock or locking sleeve to be placed fully onto the stem (e.g., failure to slide the catheter completely over the barb on the port stem). When the catheter is not advanced far enough, the barb on the port stem in combination with the cathlock or locking sleeve may not be able to hold the catheter in place in a robust way. In either of the above cases, the connection may fail due to incorrect placement of the catheter over the port stem.

One solution to overcome this problem requires the fabrication of access ports in which the catheter is pre-attached at the factory. While this practice alleviates many of the problems associated with leakage and failure due to catheter slippage, such a system severely limits the type of the catheter usable with the access port. Because port connections to catheters in this manner are permanent, if the catheter is to be shortened by trimming, the trimming must occur at the distal end of the catheter, which precludes the use of any type of specially designed tip or valve at the distal end thereof. For example, catheters utilizing a Groshong® slit valve at their distal end may not have any of the distal tips of the catheter removed without compromising the catheter. Furthermore, the cost of providing implant catheters with the desired combination of access ports, catheters, and valves at the distal end of the catheter may increase if the physician must rely on pre fabricated solutions. Moreover, pre-attached systems eliminate the option of constructing and customizing the access port and catheter combination device based on individual patient's needs.

Therefore, a port stem that is capable of ensuring a secured connection between the access port and the catheter is needed to alleviate many of the problems associated with leakage and failure due to catheter slippage. In addition, access ports with features that can assist the physician in consistent placement of catheter on the access port may improve the quality of the medical procedure as it would decrease variability in the procedure, and ensure that the catheter is placed at a location on the port stem having been previously tested to provide a secured connection.

### BRIEF SUMMARY OF THE INVENTION

Described herein is an implantable access port having a marker or indicia located on the port stem to aid the physician in placing the catheter correctly onto the port stem. In one variation, the port stem is the part of the access port extending from a housing which supports a fluid chamber. The chamber is covered by a septum and the outlet of the chamber connects to a channel within the port stem to allow fluids to flow in and out of the chamber.

A marking is provided on the port stem which indicates to the physician the proper distance to advance the port stem into the catheter for optimal connection. The port stem may have one or more barbs on its outer surface to retain the catheter on the port stem. A catheter lock or a locking sleeve may be placed over the catheter and port stem connection to secure the catheter on the port stem. Since the preferred location for the placement of the catheter may vary depending on the design of the port stem or the property of the catheter, such a marker may facilitate consistent placement of the catheter and avoid problems associated with physicians placing the catheter incorrectly (e.g., advancing the catheter to far over or failure to advance the catheter far enough), which could potentially cause in vivo leakage of fluids from the catheter, either due to detachment of the catheter from the port stem, compromise in the catheters integrity, or failure of the seal between the catheter and the port stem.

The present invention may provide an access port which can be consistently connected to a catheter that cannot be trimmed at the distal end thereof. The present invention may also provide an access ports that may be connected directly to a catheter without any intermediate member between the catheter and the port stem. In addition, the port stem may have a barb or other structural profile on its outer surface to provide positive retention of the catheter thereupon. The marking may be in a position on the port stem such that when the proximal end of the catheter is aligned with the marker the catheter fully extends over the barb thus achieving a quality seal between the port stem and the catheter. As discuss earlier, locking mechanisms may be placed around the catheter to provide additional support to maintain the connection between the port stem and the catheter.

Having a marking on the port stem to provide guidance to the surgeons on the placement of the catheter may minimize failure due to inappropriate placement of the catheter. In addition, the marking may also facilitate the surgical procedures for the implantation of the access port since the surgeon may place the catheter onto the catheter with confidence and without the need to repeatedly test and inspect the juncture to be assured that a secured connection has been achieved. Therefore, these benefits may decrease implantation time, reduce failure rate, and decrease overall cost of the procedure for implantation of the access port.

These and other embodiments, features and advantages of the present invention will become more apparent to those skilled in the art when taken with reference to the following more detailed description of the invention in conjunction with the accompanying drawings that are first briefly described.

### BRIEF DESCRIPTION OF THE DRAWINGS

In the accompanying drawings, reference characters refer to the same parts throughout the different views. The drawings are intended for illustrating some of the principles of providing a marking on the port stem of an access port to assist user with placement of a catheter and are not intended to limit the description in any way. The drawings are not necessarily to scale, emphasis instead being placed upon illustrating the depicted principles in a clear manner.

FIG. 1 is a cross-sectional view of a variation of an access port. A proximal section of a catheter is shown unassembled from the corresponding access port.

FIG. 2A is a top view of another variation of an access port having a marking on its port stem. The proximal section of a catheter to be placed over the port stem is also shown.

FIG. 2B shows the access port and the catheter from FIG. 2A in an assembled state. The proximal end of the catheter is shown closely aligned with the marker on the port stem.

FIG. 2C shown the assembled access port and catheter from FIG. 2B having a locking sleeved placed over the distal section of the port stem to secure the catheter on the port stem.

FIG. 3A illustrates a side view of yet another variation of an access port with its corresponding catheter and locking sleeve in the unassembled condition.

FIG. 3B illustrates a cross-sectional view of the access port, the catheter and the locking sleeve shown in FIG. 3A. The catheter and the locking sleeve are shown attached to the port stem of the access port.

FIG. 4A shows a cross-sectional view of another variation of an access port, where the marking is provided as an indentation on outer surface of the port stem.

FIG. 4B shows a cross-sectional view of another variation of an access port, where the marking is provided as a protrusion on the outer surface of the port stem.

FIG. 4C shows yet another variation of an access port where the marking is provided by a contrast agent or material being embedded in the port stem to provide user with a visual cue.

FIG. 5 is a plain view of another variation of a port stem where a series of individual markings which are aligned with each other are provided around port stem to provide a reference point for the user.

FIG. 6 is semitransparent view of a catheter being placed on the port stem of an access port. The port stem is shown without the housing of the access port. In this variation, the port stem marking is provided as a band. The proximal end of the catheter is to be positioned within the boundary defined by the two edges of the band.

FIG. 7 is another variation of a port stem marking comprises a band. In this variation the band-shaped marking is provided by coloring two sections of the port stem and leaving a contrasting region as the marking.

FIG. 8A is another variation of a port stem marking where the marking is provided by forming two bands on the port stem of the access port.

FIG. 8B illustrates the port stem shown in FIG. 8A having a catheter placed on the distal section of the port stem. The proximal end of the catheter is positioned such that the distal band is covered by the catheter, while the proximal band is exposed and may be visually verified by the user.

FIG. 9A shows another variation of a port stem marking where the marking is provided by a two sets of indicia aligned along the length of the port stem. In this variation, the marking is provided as indentations on the outer surface of the port stem.

FIG. 9B shows another variation of a port stem marking where the marking is provided as contrast materials being placed on the outer surface of the port stem.

FIG. 10 is a cross-section view of another variation of a port stem with a catheter secured on the port stem by a locking sleeve. In this configuration, the compressed catheter does not abut the flanges of the access port housing. In addition, a locking mechanism is provided to secure the locking sleeve to the housing.

FIG. 11 is a plan view of another variation of an access port having two chambers. In this variation, each of the chambers has an access channel in the port stem. A port stem marking is provided on the port stem to assist the user with the placement of the dual lumen catheter on the port stem. The corresponding dual lumen catheter and the locking sleeve are also shown.

### DETAILED DESCRIPTION OF THE INVENTION

The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention. Before describing the present invention, it is to be understood that unless otherwise indicated this invention need not be limited to applications in human. As one of ordinary skill in the art would appreciate, variation of the invention may be applied to other mammals as well.

A single chamber access port is used herein as an example application to illustrate the functionality of the different aspects of the invention disclosed herein. It will be understood that embodiments of the present invention may be applied in a variety of access ports (e.g., access port with two or more fluid chambers) and need not be limited to single chamber access ports described herein. In addition, the invention may be adapted for connecting catheters having a plurality of lumen to an access port having one or more fluid chambers. It must also be noted that, as used in this specification and the appended claims, the singular forms “a,” “an” and “the” include plural referents unless the context clearly dictates otherwise. Thus, for example, the term “a chamber” is intended to mean a single chamber or a combination of chambers, “a liquid” is intended to mean one or more liquids, or a mixture thereof.

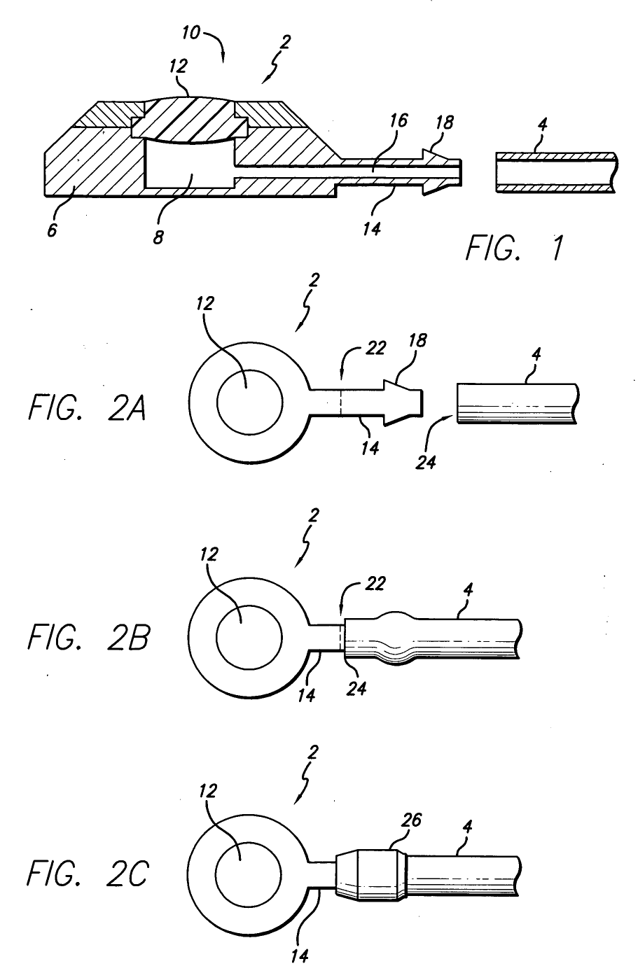

Referring to FIG. 1, one particular design variation of an access port 2 with its corresponding catheter 4 is shown in the disassembled condition. The access port comprises a housing 6 constructed of a plastic material. Within the housing 6 is a fluid chamber 8. The primary opening (i.e., the access aperture) to the fluid chamber is sealed by a septum 12. A port stem 14 extents from the housing 6 and provides and outlet to the fluid chamber. As seen in FIG. 1, a channel 16 is provided to allow fluids in the fluid chamber 8 to flow through the wall of the housing 6 and the port stem 14 to exit the access port.

The housing 6 may be comprised of various materials such as polymeric material, a combination of polymeric materials, metal or metal alloyed. In addition, the housing 6 may be configured with various shapes depending on the specific application for which the access port 2 is designed. Although, one fluid chamber 8 is shown, as one of ordinary skill in the art would appreciate, the housing 6 may be configured to support two or more fluid chambers 8. The septum 12 is configured such that it may be punctured by a non-coring needle, and re-sealed after the needle has been removed. The septum 12 may be constructed from a self-sealing polymer such as silicone rubber or latex.

Also shown in FIG. 1 is the proximal portion of the catheter 4. The catheter 4 may be made of a biocompatible rubber (e.g., silicone rubber, polyurethane), surgical tubing or other medical grade tubing commonly used for implantation. The catheter 4 may be slid onto the port stem 14. A barb 18 or other surface features or profiles (e.g., retention knob) may be provided on the outer surface of the port stem to prevent the catheter 4 from sliding off. An additional lock sleeve, crimp ring or catheter lock may be placed over the catheter and port stem connection to secure the proximal section of the catheter on the port stem. The port stem 14 may be a part of the housing formed during the manufacturing process. Alternatively, the port stem 14 may be a separate part that is connected to the housing either by the manufacturer or the user. The access ports implemented in the present invention may vary in size or geometry. In addition, the access ports may be comprised of various materials such as metal, metal alloys, or biocompatible polymeric materials.

FIG. 2A illustrates one variation of an access port having a marking 22 on the port stem. The marking 22 indicates to the physician how far to push the catheter 4 when inserting the port stem 14 into the proximal end 24 of the catheter 4. If the port stem 14 is pushed too far into the catheter 4, the marking will be covered by the distal end 24 of the catheter 4. If the catheter 4 is not pushed far enough onto the port stem 14, a space will appear between the marker 22 and the distal end 24 of the catheter 4. As shown in FIG. 2B, the catheter 4 has been placed onto the port stem 14. The catheter 4 is pushed up far enough onto the port stem 14 such that the distal end 24 of the catheter is positioned next to the marking 22. In FIG. 2C, a locking sleeve 26 is placed over the port stem 14 and catheter 4 connection to secure the distal section of the catheter 4 on the port stem. 14. The locking sleeve 26 may be a cathlock that slides over the length of the catheter 4 and over the barb 18 on the port stem 14 and maintains a compression over the distal section of the catheter 4. Other active or passive locking mechanisms that are well known to one of ordinary skill in the art may also be used to secured the catheter 4 on the port stem 14.

One example of an implantation of an access port is described below. The distal end of catheter is entered into a major vessel of the cardiovascular system of a patient and advanced therefrom, for example, into a position at the superior vena cava. After the catheter is thusly positioned, sufficient slack to allow for normal body movement without straining catheter is left in the point of entry of catheter into the vascular system. The free end (i.e., the proximal end) of the catheter is tunneled from its point of entry into the vascular system to a pocket in the tissue of a patient. The proximal end of the catheter is attached to the port stem on the access port by inserting the post stem into the catheter. The catheter's position on the port stem is adjusted so that the proximal end of the catheter is aligned with the marking on the port stem. A locking sleeve is slid over the proximal section of the catheter onto the catheter and port stem connection. The access port is secured into the pocket using sutures and may be placed in the chest wall on either the right or the left side supported by the underlying ribs. The access port is buried below the skin, and the pocket is then closed.

Once the access port is implanted inside the patient, the physician may locate the access port and its septum through tactile perception. The septum on the access port is configured such that it may be punctured by a non-coring needle, and re-sealed after the needle has been removed. Once the physician locates the septum, the physician may inject or withdraw fluids from the patient's body by inserting a non-coring needle though the skin and the septum into the fluid chamber inside the access port. The above implant procedure is only exemplary and, as one of ordinary skill in the art would appreciate, the access port may also be implanted in various other parts of the body for various medical applications. In addition, the order of assembly of the catheter and the access port may be varied during implantation. For example, the user may attach the catheter onto the access port before implanting the device into a patient's body.

The marking on the port stem may be in the form of ink, shrink wrap, plastic ridge, or etching that is machined or laser cut onto the outer surface of the port stem. Other materials or polymer markers may also be attached on to the port stem by means that are well known to one of ordinary skill in the art. As one of ordinary skill in the art would appreciate, the marking or indicia may also be implemented during the formation of the port stems. For example, indentation, protrusion or other raised or depressed profile may be molded into a polymer based stem. In addition, laser, plasma, or other heat or light treatment may also be used to mark the stem by changing the color of the plastic, titanium alloyed or other materials comprises the port stem. The marking or indicia may completely encircle the port stem at a given position along the length of the port stem. Alternatively, the marking or indicia may not completely encircle the port stem. For example, one or more dots may be provided at the desired location to indicate the proper distance of insertion along the length of the port stem.

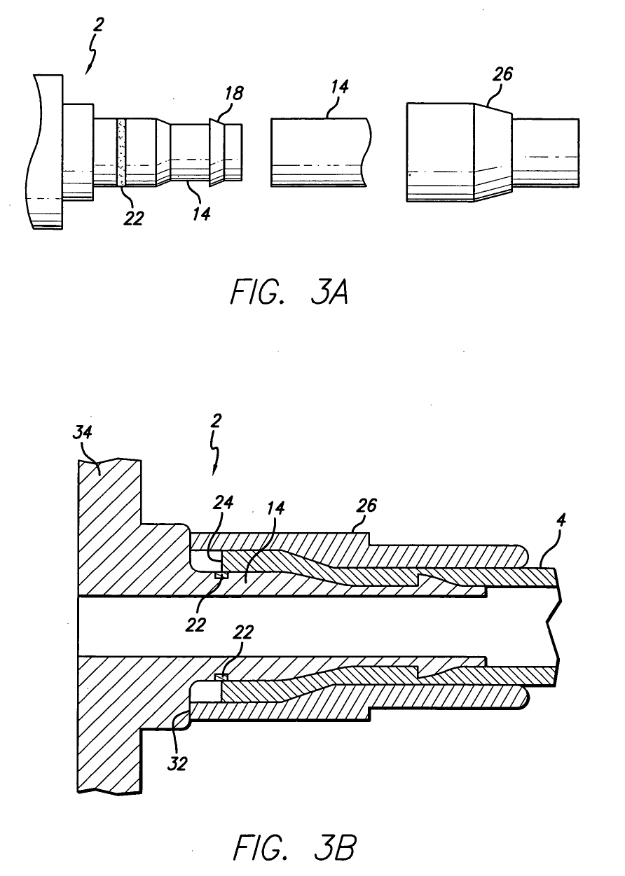

FIG. 3A illustrates another variation of the access port 2, catheter 4 and locking sleeve 26 combination. A marking 22 is located on the port stem 14 to guide the user on the proper location to place the catheter 4 so that a secured connection between the catheter 4 and the port stem 14 may be achieved. The position of the marking 22 may be determined based on various design considerations. In one variation, the location of the marking 22 is dependent on the kind of catheter 4 the access port 2 is designed to accommodate. For example, a silicon catheter tends to advance/slide on the port stem as the locking sleeve 26 (e.g., cathlock) is being placed. Thus, the position of the marking 22 takes in to account the anticipated slide of the catheter 4 when the locking sleeve 26 is later pushed on, such that after the locking sleeve 26 is placed over the catheter 4, the catheter 4 will slide into the desired position (i.e., a location where good connection may be maintained between the port stem 14 and the catheter 4). In another variation, one may place the marking 22 just over the barb 18. When the locking sleeve 26 is pushed forward over the port stem 14 and the proximal portion of the catheter 4, the catheter will slide forward into a desired location.

In yet another variation, when a catheter with a slick outer surface (e.g., a polyurethane catheter) is being implemented, it may be desirable to position the marking 22 at the optimal position on the port stem, since the catheter will not slide when the locking sleeve 26 is pushed forward over the port stem 14 and the proximal portion of the catheter 4. In another variation, two separate markings are provided on the port stem: one for silicon catheter and one for polyurethane catheter. The two markings may have characteristics (e.g., shape, color, and pattern) that allow the user to differentiate between them. For example, the marking that corresponds to the silicon catheter may be a blue circular band; while the marking that corresponds to the polyurethane catheter may be a red circular band.

In view of the disclosure herein, one of ordinary skill in the art would appreciate that the marking may be implemented to designate the optimal position for the placement of the catheter on the port stem. Alternatively, the position of the marking may take into account the sliding of the catheter that will take place when the user tries to secure the catheter on the port stem, such that after the lock sleeve is put in place, the catheter will end-up at the optimal position. The optimal location for the placement of the catheter may be dependent on the design of the port stem. In addition, the optimal location may also be dependent on the type of catheter being used. The optimal location may be determined before the manufacturing of the access port through laboratory testing, computer modeling or other methods that are well known to one of ordinary skill in the art.

For example, a prototype of a new port stem design may be fabricated for laboratory testing. The lab technician may test the prototype port stem with catheters to determine the optimal location to place the catheter on the port stem. Once this optimal location is determined, one may then integrate this information into the manufacturing process to provide a marking at the optimal location on the port stem for each of the access port being fabricated. The final product may be shipped with a corresponding instruction for user, instructing the user on the proper procedure in relying on the marking for guidance on the placement of the catheter. In addition, the instruction may also provide recommendations on the appropriate type of catheter to be implanted with the specific type of access port.

FIG. 3B is a cross-sectional view showing the access port 2, the catheter 4 and the locking sleeve 26 from FIG. 3A in an assembled condition. In this variation, the marking 22 is positioned such that when the user properly aligns the catheter 4 prior to the application of the locking sleeve 26, the final assembly will have a good connection between the three parts: access port 2, catheter 4, and locking sleeve 26. As seen in FIG. 3B, in this variation, the proximal section of the catheter 4 is evenly distributed along the length of the port stem 14, and the proximal end 24 of the catheter does not abut the edge 32 of the access port housing 34.

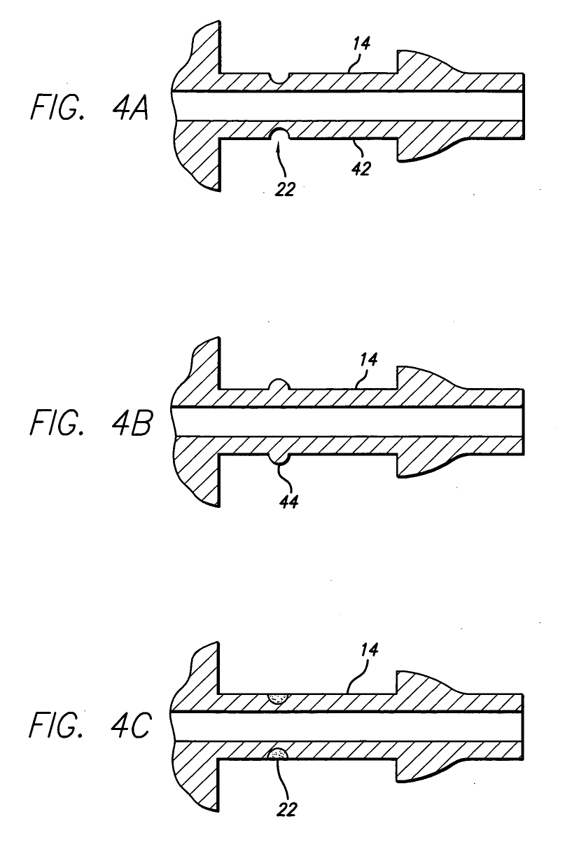

Referring to FIG. 4A another variation of a marking 22 on the port stem 14 is shown. In this variation, an indentation is provided on the outer surface 42 of the port stem 14 to serve as a visual reference. The indentation may be molded on to the port stem 14 during fabrication. Alternatively, laser or mechanical cutting tools may be implanted to cut a grove on the port stem 14. Alternatively, a raised profile 44 may be implemented on the port stem to serve as the marking, as shown in FIG. 4B. The protruded feature may be molded on the port stem 14 during fabrication or may be provided by attaching additional material onto the port stem 14. In another variation, the marking 22 is provided by embedding particles or materials into the port stem 14 as shown in FIG. 4C. In one variation, the marking 22 may completely surrounds the circumference of he port stem 14. Alternatively, the marking 22 may only partially surround the port stem 14.

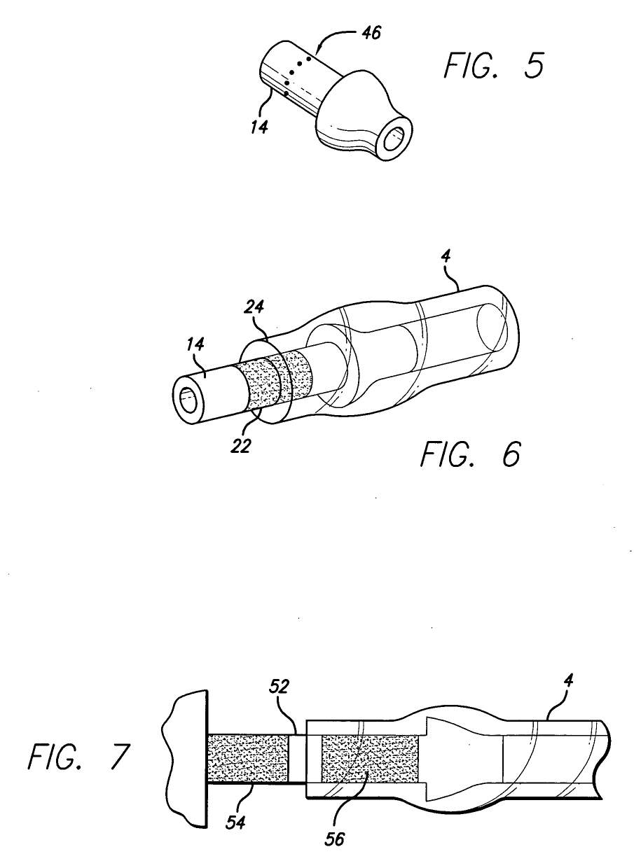

In another variation, a plurality of disconnected features 46 are placed around the circumference of the port stem 14 to serve as a marking 22, as shown in FIG. 5. FIG. 6 illustrates another variation where the marking 22 is provided as a band. In this variation, the width of the band and the location of the band is designed such that placement of the proximal end 24 of the catheter 4 anywhere within the boundary of the band may result in a secure connection between the port stem 14 and the catheter 4 when the locking sleeve is put in place. In another variation, the marking band 52 is provide by coloring two sections 54, 56 along the length of the port stem to define an un-colored section 52 as the marking, as shown in FIG. 7.

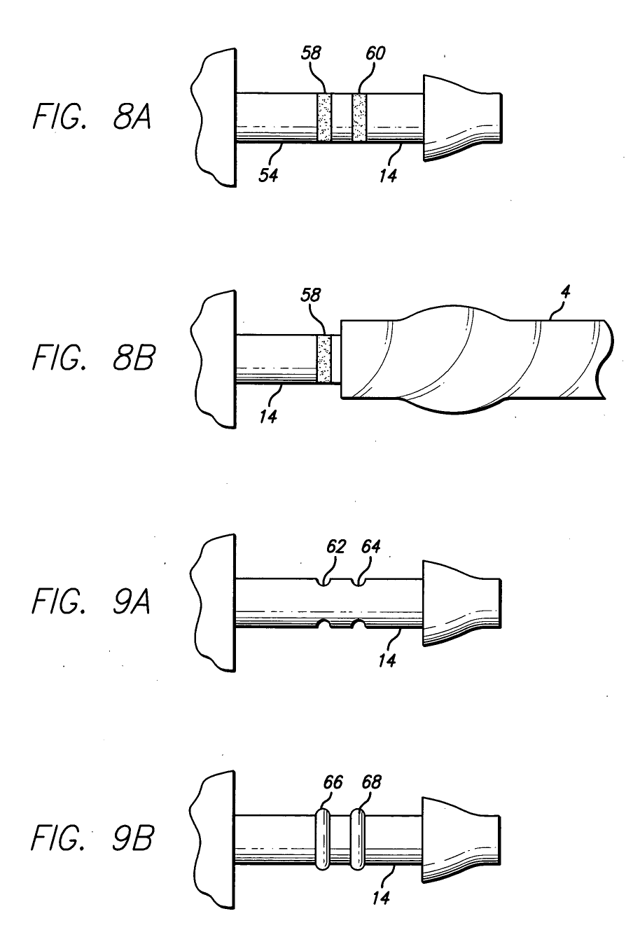

In yet another variation, the marking is provide with two separate indices 58, 60 located along the length of the port stem 14, as shown in FIG. 8A. In this variation, when the catheter 4 is placed on the catheter in the appropriate position, the indicia 60 closer to the distal end will be covered by the catheter 4, and the indicia 58 closer to the proximal end of the port stem will still be exposed to provide visual verification to the user. A locking sleeve which allows visual verification of the catheter may also be used along with the marking system to allow user to verify that the catheter is properly positioned after the assembly of the access port, the catheter, and the locking sleeve is completed. For example, the locking sleeve may be constructed of a transparent material such the user may verify the position of the catheter after the locking sleeve has been put in place. Alternative implementations of the multi-indicia marking arrangement are shown in FIG. 9A and 9B. In FIG. 9A, a pair of notches 62, 64 are implemented to provide user with a reference position. In FIG. 9B, a pair of rings 66, 68 are placed along the length of the port stem to provide such marking.

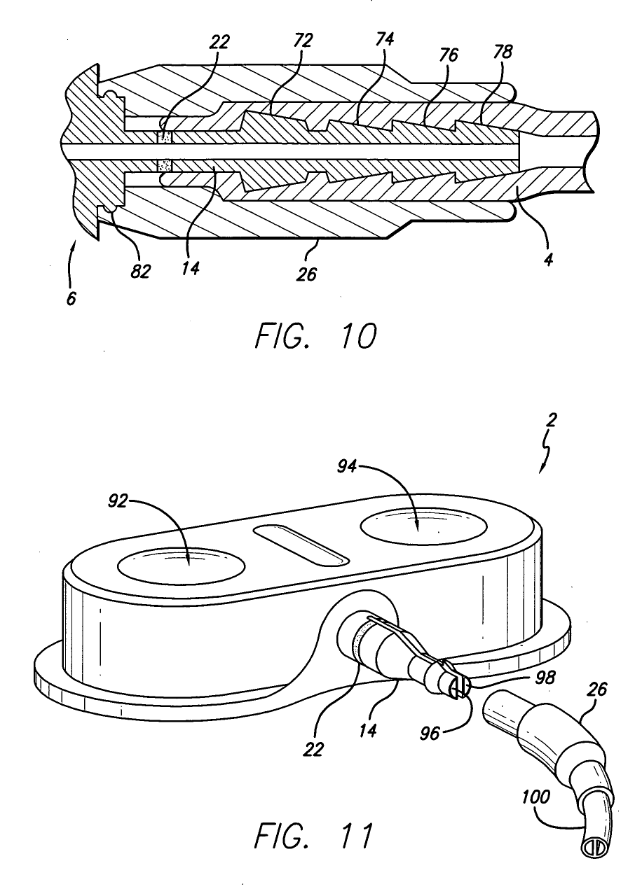

As one of ordinary skill in the art would appreciate, port stems having various profiles may be implemented with the marking system describe herein. For example, a barb may be provided on the outer surface of the port stem to prevent the catheter from sliding off. In one variation, a plurality of bars barbs 72, 74, 76, 78 are provide as shown in FIG. 10. A retention knob may also be provided at the distal end of the port stem or along the distal portion of the port stem to retain the catheter on the port stem. Threads or other surface profiles may also be provided to improve retention of the catheter on the port stem. Locking mechanisms may also be provided to prevent the locking sleeve form sliding out of position after it is placed in position. For example, as seen in FIG. 10, a notch 82 may be provided at the distal end of the locking sleeve to allow the locking sleeve to lock onto the housing 6 of the access port. Threads or other locking features may also be provided to ensure that the locking sleeve stays connected to the housing.

FIG. 11 illustrates another variation of an access port 2 where the access port 2 has more than one fluid chamber 92, 94. In the particular variation shown in FIG. 11, each of the fluid chambers 92, 94 has a corresponding outlet channel 96, 98 within the port stem. A matching dual lumen catheter 100 is provided for connection to the port stem 14. A marking 22 is provided on the port stem to guide the user on the appropriate amounts of insertion of the port stem 14 into the dual lumen catheter 100. The marking 22 may be positioned to prevent the user from over insertion of the port stem 14, which may cause bunching or poor sealing between the catheter 100 and the port stem 14. As one of ordinary skill in the art would appreciate, the marking system described herein may also be applied to access ports have three or more fluid chambers.

All publications and patent applications cited in this specification are herein incorporated by reference in their entirety as if each individual publication or patent application were specifically and individually set forth herein.

This invention has been described and specific examples of the invention have been portrayed. While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is my intent that this patent will cover those variations as well.

1. An implantable access port comprising:
a housing comprising a fluid chamber and an access aperture in fluid communication with said fluid chamber, wherein said access aperture is covered by a septum;
a port stem extending from said housing, wherein said port stem has an inner lumen forming a channel in fluid communication with said fluid chamber; and
a marking for providing guidance to a user for placement of a catheter over said port stem, wherein said marking is located on said port stem between a distal end of said port stem and a proximal end of said port stem.

2. The access port according to claim 1, wherein said marking comprises a contrast agent.

3. The access port according to claim 2, wherein said contrast agent comprises an ink.

4. The access port according to claim 1, wherein said marking comprises a contrast material.

5. The access port according to claim 4, wherein said contrast material comprises a ribbon.

6. The access port according to claim 5, wherein said ribbon comprises a metallic material.

7. The access port according to claim 5, wherein said contrast material comprises a shrink-wrap plastic.

8. The access port according to claim 1, wherein said marking is positioned on said port stem such that when the catheter aligned with said marking is compressed by a locking sleeve, a proximal end of said catheter does not abut said housing.

9. The access port according to claim 1, wherein said marking comprises an indentation on an outer surface of said port stem.

10. The access port of claim 1, wherein said marking comprises a raised profile on an outer surface of said port stem.

11. The access port of claim 1, wherein said marking comprises at least two features aligned along the length of said port stem.

12. The access port of claim 11, wherein said features are configured such that said features direct the user to place a proximal end of said catheter between said two features.

13. An implantable access port capable of being implanted beneath the skin of a patient, the access port enabling repeated, non-destructive fluid communication between the tip of a hypodermic needle piercing the skin of the patient and the proximal end of a lumen within a catheter implanted in the body of the patient coupled to the access port, said access port comprising:
an outlet stem extending from a housing of said access port, configured at a distal end thereof to receive the proximal end of said catheter, said stem enclosing a stem channel extending between a proximal end thereof and said distal end, wherein said stem channel is in fluid communication with a cavity in said housing; and
a marking positioned on an outer surface of said outlet stem, wherein said marking is located between said proximal end and said distal end of said outlet stem, and wherein said marking is configured to provide a visual reference for the placement of said catheter.

14. The access port according to claim 13, wherein said marking comprises a raised profile on the outer surface of said outlet stem.

15. The access port according to claim 14, wherein said marking comprises an indentation on the outer surface of said outlet stem.

16. A method of making an access port having a port stem marking comprising:
fabricating an implantable access port capable of being implanted beneath the skin of a patient, said access port comprising an outlet stem extending from a housing of said access port, configured at a distal end thereof to receive the proximal end of said catheter, said stem enclosing a stem channel extending between a proximal end thereof and said distal end, wherein said stem channel is in fluid communication with a cavity in said housing; and
providing a marking on said outlet stem for guiding a user on placement of a catheter over said stem, wherein said marking is located between said proximal end and said distal end of said outlet stem.

17. The method according to claim 16, wherein the act of providing a marking comprises forming an indentation on the surface of said stem.

18. The method according to claim 16, wherein the act of providing a marking comprises forming a protruding structure on the surface of said stem.

19. The method according to claim 16, wherein the act of providing a marking comprises placing said marking at a location on the stem while taking into account the amounts of sliding of the catheter when a locking sleeve is placed over the catheter.

20. The method according to claim 16, wherein the act of providing a marking comprises positioning said marking on the stem such that when the catheter aligned with said marking is compressed by a locking sleeve, a proximal end of said catheter does not abut said housing.

21. A method of connecting a catheter to an access port during implantation of said access port beneath the skin of a patient, the access port enabling repeated, non-destructive fluid communication between the tip of a hypodermic needle piercing the skin of the patient and the proximal end a lumen within a catheter implanted in the body of the patient coupled to the access port to thereby inject a fluid from the needle into the body of the patient by producing a flow of the fluid from the tip of the needle, through the access port, and along said lumen to the distal end of the catheter, comprising:
inserting a port stem of said access port into the proximal end of the catheter; and
adjusting the position of said catheter on said port stem such that the proximal end of said catheter is aligned with a marking on the port stem, wherein said marking is positioned on the port stem as a visual reference for a securing connection between the catheter and the access port.

22. The method according to claim 21, further comprising the act of placing a locking sleeve over the port stem and the proximal section of said catheter to secure said catheter on said port stem.

23. The method according to claim 22, wherein the location of said marking takes into account the amounts of sliding of the catheter when the locking sleeve is placed over the catheter.

24. The method according to claim 22, wherein said marking is positioned on said port stem such that when the catheter aligned with said marking is compressed by the locking sleeve, the proximal end of said catheter does not abut a housing of said access port.
